SEQUENCE LISTING (2 sequences)

SEQ ID NO: 1 - DNA, 24 bp, Artificial Sequence
  Oligonucleotide primer.
tgcatacaaaattgtcaagaaagg

SEQ ID NO: 2 - DNA, 23 bp, Artificial Sequence
  Oligonuceotide primer.
gggtgtatattgtggaatggcat

(19) United States
(12) Patent Application Publication
(10) Pub. No.: US 2010/0227386 A1
(43) Pub. Date: Sep. 9, 2010

(54) COMPACT OPTICAL DETECTION SYSTEM

(76) Inventors: Pavel Neuzil, Singapore (SG); Juergen Pipper, Singapore (SG); Lukas Novak, Prague (CZ)

Correspondence Address: KLARQUIST SPARKMAN, LLP, 121 SW SALMON STREET, SUITE 1600, PORTLAND, OR, 97204, US

(21) Appl. No.: 12/438,725

(22) PCT Filed: Aug. 24, 2007
(86) PCT No.: PCT/SG2007/000272

§ 371 Date: Feb. 24, 2009

Related U.S. Application Data
(60) Provisional application No. 60/839,678, filed on Aug. 24, 2006.

(51) Int. Cl. C12M 1/34; G01J 1/58

(52) U.S. Cl. 435/288.7; 250/458.1; 250/216

(57) ABSTRACT

A detection system is provided, the detection system comprising a light source that generates excitation light having a wavelength sufficient to excite a fluorophore in a sample; an excitation filter positioned along a first line along a path of the excitation light, the excitation filter transmitting the excitation light from the light source; a beam splitter positioned along the first line, the beam splitter reflecting the excitation light transmitted by the excitation filter along a second line toward a mirror positioned on one side of the beam splitter, and passing emitted light reflected along the second line; the mirror, positioned to reflect the excitation light from the beam splitter to the fluorophore in the sample along a third line, normal to both the first and second lines, wherein the mirror further reflects emitted light emitted along the third line, along the second line toward the beam splitter; an emission filter positioned along the second line, on a second side of the beam splitter; and a detector that detects the emitted light transmitted by the emission filter.

### CROSS-REFERENCE TO RELATED APPLICATION

This application claims the benefit of, and priority from, U.S. provisional patent application No. 60/839,678, filed on Aug. 24, 2006, the contents of which are incorporated herein by reference.

### FIELD OF THE INVENTION

The present invention relates generally to optical detection systems, and particularly to compact optical detection systems for detection of a fluorescent signal.

### BACKGROUND OF THE INVENTION

Lab-on-a-chip systems have been developed for various applications, such as drug discovery, pathogen detection, and others. These systems process biological or chemical samples, and provide for qualitative or quantitative detection of a target molecule or particle. Such systems use miniaturized components and are designed to be portable, allowing for sample testing in the field.

Particularly, the necessity for portable devices capable of field use to detect biological weapons, pathogens or viruses has resulted in the development of a new class of portable thermocyclers useful for polymerase chain reaction (PCR) detection of nucleic acids, including real-time PCR methods, also referred to as quantitative PCR.

Different detection techniques have been employed in these portable detection systems, including mass flow, electrochemical and optical detection methods. Optical detection methods, such as detection of a fluorescent signal, are used more frequently, due to robustness, high signal to noise ratio and sensitivity. Such methods are indispensable for applications, such as real-time PCR, capillary electrophoresis and other analytical methods.

Optical systems for the detection of fluorescent signals typically consist of the following components: a light source for emitting light at a suitable wavelength, an excitation filter to eliminate unwanted light, a dichroic mirror for the separation of the excitation and emission wavelength, an emission filter to suppress excitation wavelength, and a detector with subsequent electronics.

Commonly used light sources in optical detection systems used in laboratory devices are mercury lamps, metal halide lamps, lasers and, more recently, light emitting diodes (LEDs). Mercury lamps have high output power and broad emission spectra. Lasers also exhibit high output power and do not require an emission filter, as they are monochromatic.

LEDs are popular light sources as they can be substantially cheaper than alternative light sources. As well, LEDs are superior to lasers, due to their long lifetime. LEDs are also convenient light sources, due to the fact that the LEDs' light output can be modulated. As LEDs are only few mm in diameter as well as in length, they can be integrated into portable systems such as those used for real-time PCR.

However, as the entire un-directed fluorescence emission tends not to be fully received by the relatively small detectors typically incorporated in these devices, optical detectors with high gain tend to be required. Most popular detectors used in fluorescence optical detection systems are photo multiplier tubes (PMT), avalanche photodiodes, photon counting modules (PCM) or based on charge-coupled (CCDs) devices. These detectors can be complex, bulky or costly and usually require special operating conditions, for example, operation in complete darkness or cooling.

Thus, there is a need for an improved optical detection system for detecting fluorescent signal that is simple in design and is compact for use in hand-held portable devices.

### SUMMARY OF THE INVENTION

The present invention relates to a detection system for detecting a fluorescent signal, for example from a fluorophore contained in a sample, the arrangement of parts within the detection system being designed so that the detection system can be manufactured with suitable dimensions, i.e. a small footprint, for inclusion in hand-held devices.

The detection system includes a modulated light source, for example a LED light source, an excitation filter, a beam splitter, an emission filter, one or more focussing lenses and a light detector.

The detection system includes a conventional mirror, and the combination of the beam splitter and conventional mirror are used to reflect the excitation light beam and the emission light beam in such a manner that the light source and detector can be arranged within the same plane. Conveniently, the sample may be located in a different plane. For example, the detection system may be configured with the light source and the detector arranged substantially perpendicular to each other. Combination of the conventional mirror with a beam splitter, such as a dichroic mirror to direct the excitation and emission beams, and the resulting arrangement of light source and detector, results in a compact arrangement of the components of the detection system, making the detection system easily miniaturized and suitable for inclusion in hand-held lab-on-chip devices.

Thus, in one aspect there is provided a detection system for detecting a fluorescent signal, comprising: a light source that generates excitation light having a wavelength sufficient to excite a fluorophore in a sample; an excitation filter positioned along a first line along a path of the excitation light, the excitation filter transmitting the excitation light from the light source; a beam splitter positioned along the first line, the beam splitter reflecting the excitation light transmitted by the excitation filter along a second line toward a mirror positioned on one side of the beam splitter, and passing emitted light reflected along the second line; the mirror, positioned to reflect the excitation light from the beam splitter to the fluorophore in the sample along a third line, normal to both the first and second lines, wherein the mirror further reflects emitted light emitted along the third line, along the second line toward the beam splitter; an emission filter positioned along the second line, on a second side of the beam splitter; and a detector that detects the emitted light transmitted by the emission filter.

In another aspect, there is provided a detection system for detecting a fluorescent signal, comprising: a light source that generates excitation light having a wavelength sufficient to excite a fluorophore in a sample; an excitation filter positioned along a first line along a path of the excitation light, the excitation filter transmitting the excitation light from the light source toward a mirror; an emission filter positioned along a second line; the mirror, positioned to reflect the excitation light to the fluorophore in the sample along a third line, normal to both the first and the second lines, wherein the mirror further reflects emitted light emitted along the third line, along the second line toward the emission filter; and a detector that detects the emitted light transmitted by the emission filter.

The detection system can readily be expanded to more than one optical channel by using a multicolour light source, for example a red/blue/green (RGB) LED, and by replacing a simple single bandpass filter with a complex triple bandpass filter. Such a configuration allows for detecting three different fluorophores or fluorescent dyes simultaneously. In this case, each single colour may be individually modulated and demodulated by application of different frequencies using only one photodiode as detector, or through the use of phase-shifting. The additional channels may be used for positive, negative or internal controls, as well as for in-situ temperature monitoring.

The present detection system may be used in portable devices where miniaturization is desirable, including devices for use in real-time PCR or reverse transcription (RT)-PCR, real-time nucleic acid sequence based amplification (NASBA), real-time whole genome amplification (WGA), real-time rolling circle amplification (RCA), real-time recombinase polymerase amplification (RPA), real-time enzyme-linked immunosorbent assays (ELISAs), real-time fluorescence immunoassays (FIAs) or real-time bioluminescent and chemiluminescent assays.

Thus, in a further aspect, there is provided a thermocycler device comprising: a detection system as described herein; a sample port for receiving a sample containing a fluorophore, the sample port positioned to place the sample in line with an excitation light reflected from the detection system; a heater positioned adjacent to the sample receiving port for heating the sample; a temperature sensor connected to the heater for detecting the temperature of the heater; a fluorescent signal processor connected to the detection system for processing a fluorescent signal detected by the detection system; a user interface module for input and output of data; and a power source for powering the device.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate, by way of example only, embodiments of the present invention,

FIG. 1 is a diagram of an optical detection system, exemplary of an embodiment of the present invention;

FIG. 2 is a schematic diagram of an electronic circuit suitable for use with a detection system having multiple LED light sources, which are modulated and demodulated for detection by a single photodiode detector;

FIG. 3 is a schematic diagram of an electronic circuit suitable for use with the detection system depicted in FIG. 1;

FIG. 4 is a schematic diagram of a thermocycler device incorporating the detection system depicted in FIG. 1;

FIG. 5 is a photograph of the integrated detection system assembled in metal housing showing the location of the LED light source, the focussing lens and the preamplifier where the photodiode detector is mounted;

FIG. 6 is a graph depicting the fluorescence intensity at 25° C. obtained from experiments using the detection system of FIG. 1 to detect fluorescent signal from fluorescein;

FIG. 7 is a graph depicting a melting curve performed on amplified PCR product of the avian influenza virus HA gene, produced using a thermocycler device incorporating the detection system of FIG. 1;

FIG. 8 is photographs of embodiments of a thermocycler device incorporating the detection system of FIG. 1, without (top panel) and with (bottom panel) housing; the arrow (top panel) points to an oil-covered droplet (virtual reaction chamber), in which the PCR amplification takes place;

FIG. 9 shows cross-sectional and perspective diagrams of an embodiment of the optical detection system;

FIG. 10 is graphs of data obtained from a real-time PCR amplification using a miniaturized thermocycler device incorporating the detection system of FIG. 1 and a 6-FAM hydrolysis probe;

FIG. 11 is a graph of data obtained from a real-time RT-PCR amplification using a miniaturized thermocycler device incorporating the detection system of FIG. 1 and a SYBR Green I-based protocol to detect the H5N1 avian influenza virus;

FIG. 12 is a schematic diagram of a complete system of an embodiment of a miniaturized thermocycler device, exemplary of an embodiment of the present invention; the top panel represents the thermal management, the middle panel represents the optical signal processing setup and the bottom panel represents the control system;

FIG. 13 is a fluorescence profile and melting curve analysis of a real-time RT-PCR amplification using the miniaturized thermocycler device depicted in FIG. 11 and a SYBR Green I-based protocol to detect the H5N1 avian influenza virus; the temperature profile of a single PCR cycle is shown as inset;

FIG. 14 is a graph of a plot of the average amplitude of the fluorescent signal of the last 5 s of the extension step at 72° C. segment as a function of the cycle number and normalized against the background; and

FIG. 15 is a graph of a melting curve analysis of an amplified PCR product conducted with a heating rate of 1° C. s⁻¹.

### DETAILED DESCRIPTION

There is presently provided an optical detection system for detecting a fluorescent signal, for example from a fluorophore contained in a sample. The detection system can be readily miniaturized due to its design geometry.

The detection system may include a light source, for example a LED light source, one or more excitation filters, one or more beam splitters, one or more detection or emission filters, one or more focussing lenses and a light detector, all arranged in a compact form.

Particularly, one example of the detection system is designed so that the detection components are situated within a single plane, with a sample receiving port located outside of the plane, allowing for stacking of the detection components above or below the sample receiving port. Thus, the detection system can be designed as a compact unit for inclusion in a lab-on-a-chip hand-held devices, in which any components required for manipulation or analysis of a sample held in the sample receiving port are stacked above or below the plane of the detection components. Such design geometry, in combination with compact, lightweight components, allows for reduction in the area occupied by the detection system, and accordingly reduces the dimensions of any device incorporating the detection system.

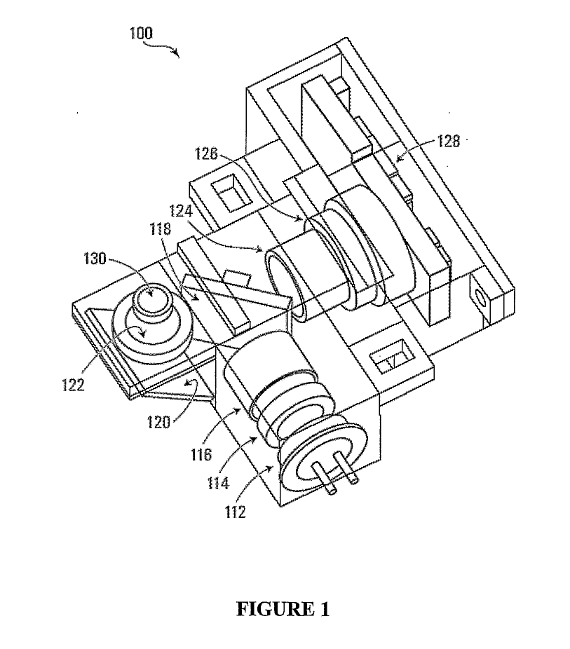

Thus, in one embodiment as depicted in FIG. 1, the detection system 100 comprises a light source 112. Light source 112 emits an excitation light beam for interaction with a sample containing a fluorophore, for generation of a fluorescent signal that is ultimately detected within the detection system as a result of interaction of the excitation light beam with the sample fluorophore.

Light source 112 may be any light source for generating light of a suitable wavelength for exciting a fluorophore contained in a sample. Examples of a suitable light source include a mercury lamp, a laser, a laser diode, a Nernst Stift, a metal halide bulb, and a light emitting diode (LED). If the detection system is to be included in a hand-held device, a LED may be used as light source 112, due to the light weight, small size and inexpensive cost of the LED.

Light source 112 may be a modulated light source. For example, light source 112 may be adjusted in frequency and/or amplitude. For example, light source 112 may be modulated, thus allowing to filter out unwanted light, for example ambient light, by demodulating the detection signal. Alternatively, light source 112 may be modulated by phase shifting of the amplitude.

For example, LEDs are commercially available, and can be chosen to emit a single colour of light or multiple colours of light. Particularly, a turquoise LED having a peak excitation wavelength between 470 and 490 nm is well suited to excite fluorophores such as SYBR-Green I, Eva Green or 6-carboxy fluorescein (6-FAM), which are the commonly used fluorescent dyes for real-time PCR applications. One example of an available LED is ETG-5CE490-15, available from ETG Corp., which emits a peak wavelength of 490 mm, in the visible light range.

Optionally, a collimating lens 114 may be arranged in alignment with light source 112 so that the excitation light beam passes through collimating lens 114. Any collimating lens suitable for focussing the excitation light beam may be used. For example, commercially available lenses such as a GELTECH™ molded aspheric lens (Thorlabs, Inc.) may be used.

Light source 112 is arranged in alignment with excitation filter 116, such that the excitation light beam generated by light source 112 passes through excitation filter 116, optionally after being collimating by collimating lens 114. Excitation filter 116 may be any filter that allows light of the wavelength required to excite the fluorophore to pass through the filter, but that blocks or attenuates other wavelengths of light. Excitation filter 116 may be a band pass filter, and such filters are known. For example, excitation filter ET470/40x is available from Chroma Technology Corporation.

Collimating lens 114 and excitation filter 116 may be combined, in order to further reduce the dimensions of detection system 100 and to improve the optical performance of the system. Such a combination lens/filter can be directly mounted against light source 112, reducing the amount of light lost from light source 112, thus increasing the efficiency of the system. See for example Bruno et al., TRAC—Trend. Anal. Chem., 1994, 13:190-198.

The optical path from light source 112 through excitation filter 116 defines a first line along which the excitation light beam travels.

Beam splitter 118 is positioned behind excitation filter 116 along the first line, and is arranged so as to reflect the filtered excitation light beam as it emerges from excitation filter 116, reflecting the beam in a path along a second line substantially perpendicular to the path along the first line from light source 112 to beam splitter 118.

Beam splitter 118 is chosen such that it reflects light of the wavelength of the excitation light beam, but that light of the wavelength of the emission light beam is able to pass through the beam splitter. Beam splitter 118 may be any suitable beam splitter, and may be, for example, a dichroic mirror. Typically, a dichroic mirror will reflect light having a wavelength shorter (or longer) than a given wavelength and transmit light having a longer (or shorter) wavelength than the given wavelength. In a particular example, beam splitter 118 may be dichroic mirror T495LP, available from Chroma Technology Corporation.

Conventional mirror 120 is arranged inline with beam splitter 118, and angled in a manner so as to reflect the excitation light beam in a path substantially perpendicular to the incident excitation light beam along a third line, the third line thus being normal to the plane defined by the first line and second line. The excitation light beam is thus reflected from the beam splitter 118, towards sample receiving port 130, which is in optical communication with a fluorophore that is to be detected, for example a fluorophore contained in a sample.

Thus, as described and as shown in FIG. 1, light source 112, beam splitter 118 and conventional mirror 120 all lie generally within the same plane. The components are aligned so that excitation light beam, once generated by light source 112, travels within this plane until reflected by conventional mirror 120, in a direction substantially perpendicular to such a plane.

A focussing lens 122 is arranged to focus the excitation light beam as reflected from conventional mirror 120 as it travels to a location at which a fluorophore that is to be detected is to be positioned, within sample receiving port 130.

Depending on the device in which detection system 100 is included, sample receiving port 130 may comprise a sample chamber designed to accept a sample such as a liquid, or to accept a tube, glass slide or other transparent container or surface holding a sample, the sample potentially including a fluorophore that is to be detected. Thus, detection system 100 may be used by immersing at least sample receiving port 130 in a sample, or detection system 100 may be used by placing a sample or a container or surface holding a sample into sample receiving port 130.

Thus, sample receiving port 130 is located above or below the plane defined by the light source 112, beam splitter 118 and conventional mirror 120.

When the excitation light beam interacts with a fluorophore that is to be detected, the fluorophore will absorb light from the excitation light beam, and emit light having a different wavelength from excitation light beam, usually having a longer wavelength. The emitted light, referred to as the emission light beam, travels back to conventional mirror 120, and is then reflected from conventional mirror 120 at an angle substantially perpendicular to the path from the fluorophore to conventional mirror 120, back to beam splitter 118. Since beam splitter 118 is chosen to transmit light having a wavelength of the emission light beam, the emission light beam passes through beam splitter 118, rather than being reflected back towards light source 112.

An emission filter 124 is positioned on a second side of beam splitter 118, opposite mirror 120. Emission filter 124 is any filter that allows light having the wavelength of the emission light beam to pass through the filter, but that attenuates or substantially blocks other wavelengths of light. Emission filter 124 may, for example, be a band pass filter. For example, emission filter ET525/50m is available from Chroma Technology Corporation.

A detector 126 is aligned behind emission filter 124, positioned to capture the emission light beam filtered by emission filter 124. Detector 126 may be any detector that can detect a fluorescent signal having the expected frequency, for example photo multiplier tube (PMT), a photon counting module (PCM), a photodiode, an avalanche photodiode or a charge-coupled (CCD) device. Particularly, the detector may be a photodiode. Photodiodes are commercially available, including silicon photodiode BPW21 available from Siemens Inc.

In order to adequately detect an emitted fluorescent signal, a detector having high gain may be employed. Alternatively, amplifier 128 may be included in detection system 100, to amplify low amplitude current generated by detector 126, particularly when detector 126 is a photodiode. Amplifier 128 is thus in communication with detector 126.

Amplifier 128 may, for example, be a lock-in amplifier, which modulates/demodulates the signal from detector 126. Miniaturised lock-in amplifiers are known, and have been described for example in Hauser et al., Meas. Sci. Technol., 1995, 6: 1081-1085.

In the above described embodiment, the path of the excitation light beam from light source 112 to beam splitter 118 along the first line is substantially perpendicular to the path from beam splitter 118 to conventional mirror 120 along the second line. However, it will be appreciated that this angle may be an acute or obtuse angle, provided that there is sufficient space for positioning of the light source and the detector about the beam splitter, and provided that the various light paths do not interfere with each other. It will further be appreciated that adjustment of the angle between the first line and the second line will require adjustment of all of the necessary components of detection system 100 so that the excitation light beam and the emission light beam are directed to the appropriate components as described above.

Conveniently, the fluorophore to be detected in the sample conveniently lies in a plane that does not intersect with the light beam from/to beam splitter 118. In this way, a sample may be placed in sample receiving port 130 in a plane above or below the optical path of incident and reflected beams. This design geometry allows the overall size of detection system 100 to remain small, by moving the sample receiving port 130 to a volume of space above or below the remaining components in detection system 100. This arrangement is also convenient in that sample port 130 may be located near or adjacent to any device components required to manipulate a sample without interfering with detection system 100, when detection system 100 is included in a device such as lab-on-a-chip hand-held devices.

As well, as mentioned above, the detection system may be expanded to include more than one optical channel, which may be useful for detection of multiple fluorophores within a single sample, or for monitoring of internal controls within a sample.

For example, a multicolour light source such as a red/blue/green (RGB) LED could be used as the light source, with replacement of the single band pass excitation and emission filters with multiple bandpass filters. Alternatively, more than one single LED could be used, the single LEDs either all having the same frequency range or colour, or each having a different frequency range or colour.

Crosstalk or interference between different optical channels may be suppressed by individually modulating/demodulating each optical channel, for example by applying different modulation frequencies. Thus, each light source or optical channel may be modulated at a unique frequency, with its own demodulator.

Only one photodiode would be needed as detector, as the different wavelength of fluorescent signals can be individually modulated and demodulated by applying different frequencies using a single photodiode.

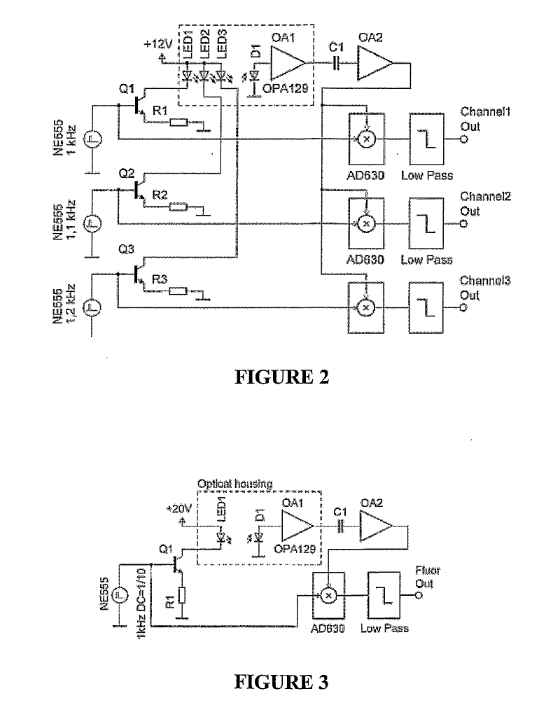

FIG. 2 depicts an electronic circuit for a triple optical channel system, each optical channel modulated at a unique frequency, and having a separate demodulator for each optical channel.

FIG. 3 is a simplified schematic diagram of an electronic circuit for optical fluorescent excitation and detection using detection system 100. The light source 112 (light emitting diode LED1) is powered by current pulses generated by a pulse voltage generator, which is converted into current by the transistor Q1. The emitted light signal detected by detector 126 (photodiode D1) is converted into a voltage by the operational amplifier OA1. Its output voltage is amplified by the operational amplifier OA2 and filtered by a demodulator AD630, which is followed by a low pass filter.

Although the above described embodiment includes a beam splitter, if the excitation light beam and the emission light beam are sufficiently different so as not to optically interfere with each other, the beam splitter may be omitted from the detection system.

In such an embodiment, the excitation light beam travels along a path along a first line through the excitation filter to the mirror, and is reflected from the mirror through the focussing lens to the sample port along a path normal to the first line. The emission light beam passes from the sample port back to the mirror where it is reflected by the mirror towards the emission filter and inline detector, which lie along a second line, also normal to the path from the mirror to the sample port, which lies along a third line. For example, the paths along the first line and the second line may lie substantially along the same line, or may be separated by a small angle, with the detector located beside the light source.

Detection system 100, due to the design geometry, is readily miniaturizable, since the components may be arranged in a very compact manner, for example where detection system 100 involves the combined use of a beam splitter and a conventional mirror to direct the various light beams through the appropriate filters, lenses and detectors. Using small, lightweight components such as an LED light source and a photodiode detector also allows for reduction of the size and weight of the detection system. Thus, in various embodiments, the detection system can be manufactured to have dimensions of about 30 mm×30 mm×11 mm.

Such a compact and lightweight detection system is suitable for inclusion in lab-on-a-chip devices, including portable hand-held devices. Thus, as stated above, the optical detection system is suitable for use in detecting fluorescent signal generated in a variety of analytical techniques, including real-time PCR or RT-PCR, real-time nucleic acid sequence based amplification (NASBA), real-time whole genome amplification (WGA), real-time rolling circle amplification (RCA), real-time recombinase polymerase amplification (RPA), real-time enzyme-linked immunosorbent assays (ELISAs), real-time fluorescence immunoassays (FIAs) or real-time bioluminescent and chemoluminescent assays.

Real-time PCR techniques have been developed based on fluorescence detection of a fluorophore covalently bound to or interacting with a PCR product. Compared to conventional PCR, such methods provide additional information regarding the number of initial copies of a target gene in the test sample. Due to recent infection disease outbreaks, such as SARS and the current threat of Avian Influenza virus (H5N1), a truly portable real-time PCR and/or RT-PCR-based device is in high demand.

Thermocycler devices (PCR devices) have been successfully miniaturized using micro-machining technology, in attempts to bring down the running costs of PCR analytical methods, as well as to make PCR analysis portable. A portable PCR device was first reported in 1994, and further advancements have been made by the original inventors (Northrup et al., Anal. Chem., 1998, 70: 918-922), as well as other groups (Higgins et al., Biosens. Bioelectron., 2003, 18: 1115-1123).

Nevertheless, a typical real-time PCR fluorescence detection system is still based on a mercury lamp or a laser for excitation and a photomultiplier tube (PMT) or a CCD device as a detector, making portable PCR devices rather complex. As well, such devices tend to be relatively costly and have high power demands, and require that the PCR amplification be performed in the absence of light in order to avoid interference of ambient light with a detected fluorescent signal.

Thus, there is also presently provided a miniaturized thermocycler device, suitable for performing real-time PCR, including real-time RT-PCR. The miniaturized thermocycler device may be portable, and in some embodiments, may be a hand-held thermocycler device.

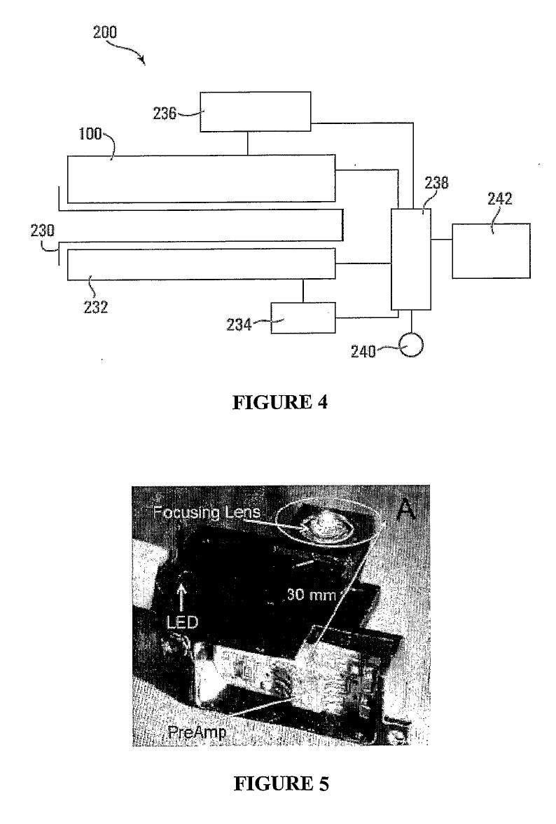

As seen in FIG. 4, in one embodiment, the thermocycler device 200 comprises detection system 100, as described above.

Thermocycler device 200 also includes a sample receiving port 230 for receiving a sample or a mixture in which PCR is to be conducted. Sample receiving port 230 is dimensioned to receive a suitable container or surface in or on which a PCR amplification is to be conducted. For example, sample receiving port 230 may be of suitable size and shape to receive a thin-walled tube, or a glass slide.

Sample receiving port 230 is arranged to receive the sample in a position so that the excitation light beam reflected from the conventional mirror is transmitted to the sample in sample receiving port 230, and the emission light beam emitted from a fluorophore contained in the sample is transmitted back to the conventional mirror contained within detection system 100.

Thermocycler device 200 further includes a heater 232 for heating the sample to the various required temperatures in the course of conducting a real-time PCR cycle. Heater 232 is adjacent to the sample receiving port 230, to allow for heating of the sample received by sample receiving port 230. Heater 232 may be in the form of, for example, heating plates or a thin film heater, such as a gold thin film.

Coupled to the heater 232 is a temperature detector 234 to measure the temperature of heater 232 at the various stages of the PCR cycle. The temperature detector 234 may be, for example, a resistance temperature detector.

The thermocycler device 200 also includes a fluorescent signal processor 236 for receiving the fluorescent signal from detection system 100, and for processing the signal for output to a user interface.

The thermocycler device 200 may optionally further include an integrated controller 238, which controls the detection system 100, the heater 232 and temperature detector 234, and which receives information from the fluorescent signal processor 236. Alternatively, if the thermocycler device is not a hand-held device, the device may be controlled by a remote controller, for example, a personal computer, not included in the device.

Thermocycler device 200 also includes a power source 240, for providing the necessary power to various components of the detection system 100, heater 232, temperature detector 234, fluorescent signal processor 236, and controller 238. If thermocycler device 200 is a hand-held device, power source 240 may be a battery.

Thermocycler device 200 also includes a user interface module 242 for receiving input from and delivering data to a user, for example a touch-screen, or other user interface command input module, for example a keyboard and display screen.

Various embodiments of the above described detection system and thermocycler device are described in the following non-limiting examples.

### EXAMPLES

### Example 1

A miniaturized fluorescence system with dimensions of 30 mm×30 mm×11 mm was designed and tested.

The properties of the detection system were determined by measuring a dilution series of the fluorescence dye fluorescein. The detection limit was found to be 1.96 nmol/L, which is more than sufficient for applications like real-time PCR.

The optical detection system has two sections: excitation and detection. The excitation section included a turquoise color LED model ETG-5CE490-15 (ETG Corp) as a light source. The LED has a peak emission wavelength of 490 nm with a luminous intensity of 6 cd (candela) and a viewing angle of 15°. Power losses were observed over the optical path, due to the viewing angle of the light source. Therefore, to collimate the light, the top of the LED plastic cover was cut by vertical milling 0.5 mm from the LED chip. The cut surface was then flattened with aluminum oxide abrasive waterproof paper and polished with a conventional diamond paste.

GELTECH™ molded glass aspheric lenses (Thorlabs, Inc.) were used for both collimating the LED light and focussing at the sample. The lenses have a diameter of 6.35 mm, a focal length of 3.1 mm, and a numerical aperture (N.A.) of 0.68. Collimated light from the first lens was filtered by exciter ET470/40x (Chroma Technology Inc.), reflected by the dichroic mirror T495LP, diverted perpendicularly by a conventional mirror and focussed at the sample of interest by the second lens, thereby forming a circular shape of excitation light with a diameter of 480 μm.

The detection path started with collection of emitted fluorescent light from a sample by the second lens. The emitted light passed through the dichroic mirror, was filtered by emission filter ET525/50m and collected by a silicon photodiode BPW2I (Siemens, Inc.). The radiant sensitive area of the diode is 7.34 mm² with a quantum yield of 0.8, resulting in an optical sensitivity of 10 nA/lx (nanoamperes per lux). The corresponding current generated by the photodiode (photocurrent) was processed by an operational amplifier placed next to the photodiode.

The components of the system were mechanically connected to each other to form a stable and compact system, using a conventional housing approach. The housing for all optical components including the amplifier was designed in SolidWorks 2006 program (Solid Works Corp.). The size of the housing was 30 mm×30 mm×11 mm (w×l×h). The housing was then manufactured by a computer numerical control (CNC) vertical milling method from an aluminum alloy AA 6060 and electrochemically blackened to suppress unwanted internal reflections.

FIG. 5 is a photograph of the integrated detection system assembled in metal housing showing the location of the LED light source, the focussing lens and the preamplifier where the photodiode detector is mounted. The filters and collimating lens are not visible in the photograph.

The optical power attenuation of all components was measured in both the excitation and emission direction, as shown in Tables 1 and 2.

TABLE 1
Measured optical properties of the excitation components of the
fluorescence detection system

| Optical component | | Relative Power |
|---|---|---|
| (excitation) | Transmission (%) | (%) |
| LED (blue band) | | 100 |
| Collimating lens | 91 | 91 |
| Blue excitation filter | 95 | 86 |
| Dichroic mirror | 99 | 86 |
| Conventional mirror | 94 | 80 |
| Focussing lens | 91 | 73 |
| Optical path | 69 | 51 |
| Total | | 51 |

TABLE 2
Measured optical properties of the detection components of the
fluorescence detection system

| Optical component | Transmission | Relative Power |
|---|---|---|
| (detection) | (%) | (%) |
| Sample (green band) | | 100 |
| Lens | 91 | 91 |
| Mirror | 95 | 86 |
| Dichroic mirror | 98 | 85 |
| Green emission filter | 97 | 82 |
| Optical path | 68 | 56 |
| Total | | 56 |

The overall optical transmission of the fluorescence detection system was found to be 51% and 56% in excitation and emission directions. Most of the components showed an efficiency of 90% or higher. The system's optical performance could be further improved by combining lenses with filters and mounting them directly to the LED and the photodiode. In addition to the reduction of number of optical interfaces, such an approach would allow for a more compact design, increasing the transmission along the optical path.

Generally, the amplitude of a photocurrent is low, and thus a technique to improve the signal-to-noise ratio was implemented. For previous detection systems, a direct approach based on high sensitivity devices has been described (Rovati and Docchio, Rev. Sci. Instrum., 1999, 70: 3759-3764). Here, a ‘lock-in’ amplifier was used, which modulates/demodulates the signal in a signal processing chain (refer to FIG. 2) (see Scofield, Am. J. Phys., 1994, 62: 129-133). The light source (LED) is first modulated at frequency f. The demodulator works as a narrow band pass filter, allowing only signals at frequency f to pass, resulting in a high signal-to-noise ratio and immunity to ambient light.

The turquoise color LED was powered by current pulses with frequency of 1 kHz, duty cycle of 10% and current amplitude of 100 mA. These pulses were generated by a single timer NE555 integrated circuit (ST Microelectronics, Inc.), followed by a standard bipolar transistor to achieve the desired current supplying the LED. Generated light passed through the optical system as described above and the emitted fluorescent light was detected by the photodiode.

The photocurrent generated by the photodiode was converted into a voltage (I/V) by ultra low bias current operational amplifier OPAI29 (Burr Brown, Inc.) with 3.3 MΩ resistor in a feedback loop. It converted each mA of photocurrent into 3.3 mV of output voltage.

The amplifier output voltage was processed by a simple high pass filter and amplified with the gain of 100 by a second stage operational amplifier OA2. The high pass filter eliminated the DC component of the signal, which is necessary for a proper function of the lock-in amplifier. Additionally, this filtering process also eliminated a possible saturation of the OA2 due to ambient light.

The output of the OA2 was then processed by a demodulator AD630 (Analog Devices, Inc.), which used the pulses powering the LED as a reference. The demodulator output was filtered by a low pass filter of 4^{th} order. This configuration of lock-in amplifier worked as a filter with band pass width of 1.5 Hz around the frequency f of reference LED signal. Due to the narrow band pass the system is relatively insensitive to ambient light and other noise contributors.

To demonstrate the capability of the fluorescent system a set of experiments was performed using different concentration of fluorescein. Fluorescein is one of the most popular fluorescent dyes used for biological and biochemical applications, and here exhibited negligible bleaching effect under the conditions used in the following described experiments.

A 1 μL droplet containing different concentrations of fluorescein was placed on top of a perfluorinated glass substrate, which was mounted in the focal plane of the miniaturized fluorescence detection system. As reference, a control sample containing only de-ionized (DI) water was used.

To estimate the probed volume, droplets of different volumes ranging from 5 μL down to 0.5 μL were initially used. It was observed that the amplitude of the fluorescent signal was not affected by the droplet size, and therefore it was assumed that the probed volume was smaller than 0.5 μL.

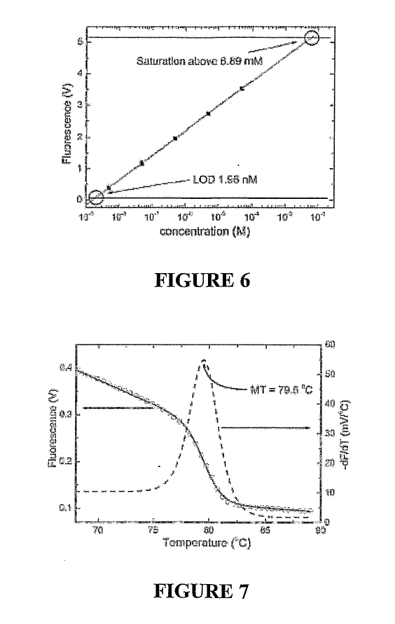

The dilution series started from a concentration of 50 μM down to 5 nM. The amplitude of the fluorescence signal was plotted as a function of the concentration in logarithmic scale (see FIG. 6). The background noise of the detected system had a value of 62.5 mV. By extrapolation the limit of detection (LOD) of fluorescein was found to be 1.96 nM.

FIG. 6 demonstrates the detection limit using a dilution series of fluorescein in water conducted at 25° C. Solid black squares are mean values of six individual measurements for the respective concentration; error bars represent the standard deviation; the solid line is a linear regression (r²=0.999) to the mean values; the solid horizontal line denotes the background of 62.5+/−1.4 mV. Three times signal-to-noise ratio (SNR 3) is 4.2 mV. The intersection of the linear regression with the background including SNR 3 indicates the LOD of the miniaturized fluorescence detection system, which is 1.96 nM. Saturation of the detector at 5.2 V determines the upper detection limit, which corresponds to a concentration of 6.89 mM.

The sensitivity of the detection system could be increased by incorporation of an avalanche photodiode. However, this solution would be more costly and the electronics require a more complex design. Established chip-based capillary electrophoresis systems based on laser induced fluorescence (LIF) typically reach 1 pM LOD. The device described in this contribution has a LOD value 1000 times higher. Nevertheless, its sensitivity is sufficient to be used for real-time PCR applications as a typical commercial PCR system based on a PMT13 has a sensitivity limit of around 5 nM.

To demonstrate the detection system's applicability, the fluorescence detection unit was integrated with a PCR chip, creating a miniaturized real-time thermocycler and performed a melting curve analysis of the PCR products (see FIG. 7) in a 1 μL volume. The sample was covered with 3 μL of a mineral oil to prevent evaporation.

FIG. 7 shows the result of a melting curve analysis after performing a real-time PCR of the HA gene of the avian flu virus H5N1 using EvaGreen (Biotiom, Inc.) as intercalator. A nonlinear fitting (solid line) of the raw data (open circles) based on a sigmoidal function was performed. Its negative derivative (dashed line) indicated a half melting temperature of 79.5 uC, which was close to that measured by a commercial thermocycler (79.8° C. by DNA ENGINE OPTICON 2™ from NJ Research, Inc.).

### Example 2

A miniaturized economical real-time PCR made of micro-machined silicon was made, incorporating the above-described optical detection system.

Here, the compact, autonomous real-time RT-PCR device is described, having dimensions of 7 cm×7 cm×3 cm, with a weight of 75 g, or in a second embodiment, dimensions of 10 cm (diameter)×6 cm (height), with a weight of 150 g.

The PCR unit is integrated with a miniaturized fluorescence detection system and all the electronics necessary for the system's operation. The turquoise light emitting diode (490 nm peak excitation wavelength) is powered by current pulses with a peak amplitude of 100 mA. Photocurrent detected by a photodiode is processed by a lock-in amplifier making the optical system independent of ambient light.

A 12 Ah battery can be used to power the thermocycler device for up to 12 hours, as the consumption of the device is only 3 W. The compact size of the thermocycler device and its power consumption assure its portability.

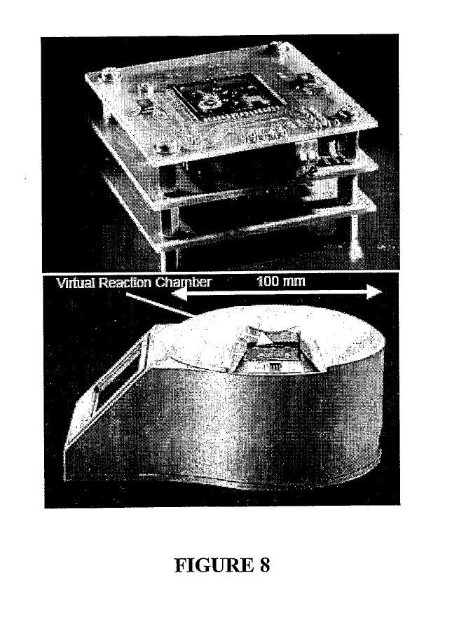

FIG. 8 shows photographs of the thermocycler device; the arrow (top panel) points to an oil-covered droplet, in which the PCR takes place.

The real-time PCR system consists of three or four printed circuit boards (PCB) linked by connectors.

The top PCB hosts a micro-machined PCR chip, which contains a thin film gold heater and temperature sensor. The optical detection system (described above) is attached beneath the PCR chip on this board. A light emitting diode (LED) with a peak emission wavelength of 490 nm is used as a light source along with a photodiode as a light detector. Light was filtered within the detection system using a fluorescein isothiocyanate (FITC) filter set.

The LED inside the optical detection system is powered by current pulses with an amplitude of 100 mA, a frequency of 1 kHz and a duty cycle of 10%. The fluorescence signal from the photodiode is filtered by a high pass filter, amplified 108 times and fed into a lock-in amplifier. Therefore, the detection system can operate under ambient light.

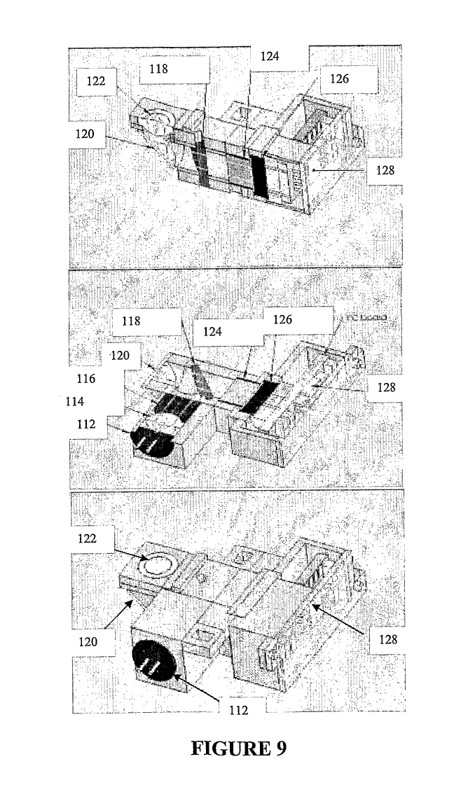

The cross-section of the optical unit is shown in FIG. 9, which depicts cross sections of the optical detection system. The light is generated by a 490 nm wavelength LED, passes through a blue filter, is diverted by a dichroic mirror and is focussed on the PCR sample (inside the droplet, see above). The fluorescent light emitted from the sample is collected by the lens, passes through the dichroic mirror and a green filter, and is detected by a photodiode.

Signal processing for the fluorescent unit is conducted at the second PCB, which contains analogue circuits for thermal management and fluorescence data processing.

The temperature of the PCR system is measured by an integrated resistance temperature detector (RTD) type of sensor connected to an AC-powered Wheatstone bridge. The signal from this bridge is amplified and demodulated to provide a DC value for temperature feedback.

The PCR temperature is controlled by modulating the amplitude of dissipated power within the heater using a proportional-integral-derivative (PID) controller.

The third PCB is connected to a single battery or a charger and generates all the necessary power for the analog and digital blocks on the other boards.

The thermocycler device may be controlled by a computer, for example equipped with a LABVIEW™ system.

Alternatively, a fourth board containing a single chip controller can be used, for example model MC56F8013 (Freescale Electronics, Inc.), making the entire thermocycler device totally autonomous. Communication with the autonomous controller is via touch screen display, on which the results are also shown.

PCR amplification was conducted on a disposable microscope cover slip. The performance of the device was demonstrated by performing a 50-cycle PCR amplification in 15 min, making the device practical for field-use PCR analysis.

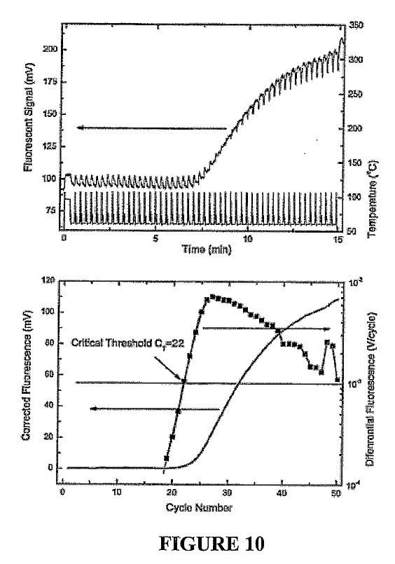

The real-time PCR data is shown in FIG. 10. A 6-FAM hydrolysis probe-based PCR amplification was performed to demonstrate a fast real-time thermocycler device. A 50-cycle reaction was conducted in less than 15 min.

FIG. 10: PCR real-time fluorescence intensity data (red) and thermal (blue) profile (top panel). The extracted data (bottom panel) shows a critical threshold of 22 cycles, which is in agreement with the result obtained from a commercial thermocycler.

### Example 3

The portable thermocycler device was tested for the genetic analysis of an infectious disease. RT-PCR performance of the thermocycler device was demonstrated by detection of RNA isolated from the avian influenza virus (H5NI) using the RNA Master SYBR Green I RT-PCR Kit (Roche, Inc.) with PCR primers developed at the Institute of Molecular and Cell Biology of Singapore.

The reverse transcription was performed at 61° C. for 2 min and 30 s, followed by a hot start at 95° C. for 30 s. Amplification over 50 PCR cycles was carried out as follows: 3 s at 95° C. (denaturation), 15 s at 50° C. (annealing) and 20 s at 72° C. (extension). Once the PCR cycling was finished, melting curve analysis was conducted with a transition rate of 1° C. s⁻¹. The total time necessary to detect the viral RNA was 14 min.

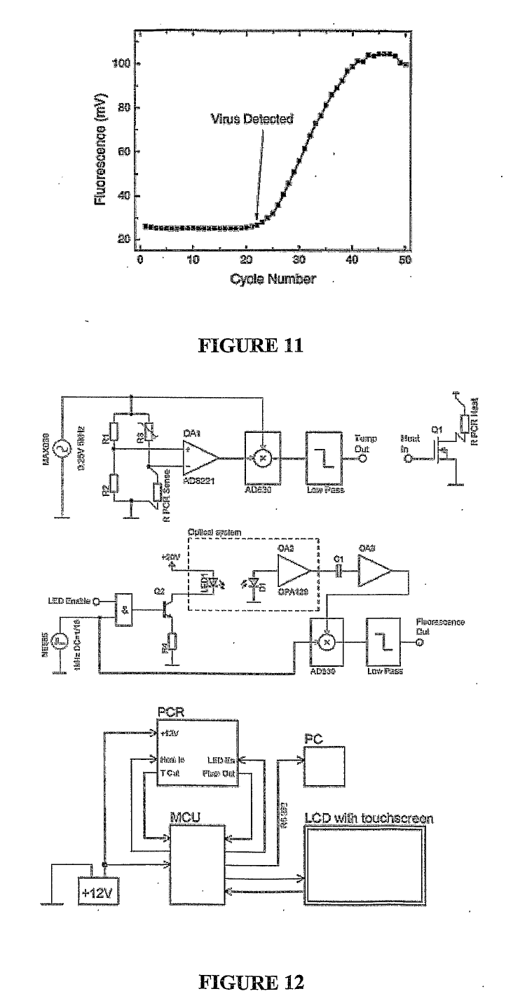

FIG. 11 shows the results of real-time RT-PCR using the present miniaturized thermocycler device to detect the H5N1 virus. The critical threshold for detection was found to be 22 cycles, which corresponds the detection time of 14 min.

### Example 4

Here, PCR amplification using the present thermocycler device is described. The volume of a PCR sample used in these methods is between 100 mL and 5 μL, routinely being 1 μL. The PCR is conducted on a disposable microscope glass cover slip. The present device is capable of detecting infectious agents like the HPAI (H5N1) virus in 20 min using a SYBR Green I-based RT-PCR technique.

Thermocycler and thermal management: The micro-machined silicon PCR chip described above was used. The thin film heater and the resistance temperature detector (RTD) were integrated into the silicon structure. The silicon chip was soldered to a PCB together with a TSic™ chip (Innovative Sensor Technology, Switzerland) used as a reference temperature sensor (calibrated with a precision of 0.05° C.; this reference temperature sensor is used to calibrated the RTD sensors of the PCR chip). All components for the thermal management were placed on a second PCB just below the first PCB containing the PCR chip.

The RTD sensor was connected via a balanced Wheatstone bridge. An internally generated sinusoidal-shaped AC signal with an amplitude of 0.25 V was applied. The bridge output was amplified by an operational amplifier AD8221 (Analog Devices, Inc.) with the gain set to 500. The amplifier output signal was then processed by a demodulator AD630 (Analog Devices, inc.) followed by a low pass filter of the 3^{rd} order.

The above setup resulted in temperature sensitivity of about 30 mV/° C. and provided no DC drift, since the bridge was powered by an AC signal. Low amplitude of the bridge bias also reduced the self-heating effect originated by the dissipated Joule heat within the PCR chip to an acceptable level of 0.2 mW.

The PCR heater was powered by a pulse-width-modulated (PWM) signal by the PID controller. The low-to-high power conversion was made possible by a power MOSFET transistor.

FIG. 12 is a block diagram of the complete system of the thermocycler device. The top panel represents the thermal management, the middle panel represents the optical signal processing setup and the bottom panel represents the control system.

Fluorescence detection system: The metal housing containing the present optical detection system for fluorescence signal detection was attached to the PCB containing the PCR chip.

Power supply and control: In order to simplify the system operation and make it user-friendly, an additional PCB was used, containing voltage generators of +12 V, −12 V, +5 V, −5 V and +3.3 V, all from a single power source between 12 V and 24 V. This PCB was linked to the analog PCB via a direct connector.

The system was controlled from a LabView program running on a PC computer, but could be designed having an integrated control system based on a touch-screen panel together with a single chip controller, for example using model MC56F8013 (Freescale, Inc.).

The performance of the thermocycler device was verified by the real-time detection of an in vitro transcribed HA segment of the H5N1 virus. The RT-PCR was set up using the LIGHTCYCLER™ RNA Master SYBR Green I One-Step RT-PCR Kit from Roche, Inc.

Sample preparation: The reaction mix was prepared by adding 1.3 μL of 50 mM Mn(OAc)₂, 0.6 μL of each forward and reverse primers with a final concentration of 0.2 pM and 7.5 μL of the LIGHTCYCLER™ RNA Master SYBR-Green I. Primers were developed by the Genome Institute of Singapore (GIS) and were validated with clinical samples of HPAI during the Southeast Asian outbreaks in 2004 and 2005. The sequences of the primers used are: forward primer 5′-TGCATACAAAATTGTCAAGAAAGG-3′ (SEQ ID NO.: 1); reverse primer 5′-GGGTGTATATTGTGGAATGGCAT-3′ (SEQ ID NO.: 2). A RNA template of 2×10⁶ copies in 10 μL was added to the reaction mix to the total volume of 20 μL immediately before starting the reaction. The final template concentration was 10⁵ copies μL⁻¹.

RT-PCR protocol: 1 μL of the sample RT-PCR mixture was transferred to a microscope cover slip on top of the thermocycler and covered with 3 μL mineral oil to prevent evaporation.

The reverse transcription was performed at 61° C. for 5 min followed by a ‘hot start’ at 95° C. for 20 s. 50 PCR cycles were carried out according to the following thermal protocol: 4 seconds at 95° C. (denaturation), 20 s at 50° C. (annealing) and 10 s at 72° C. (extension). The total time required for a single cycle was 34 s.

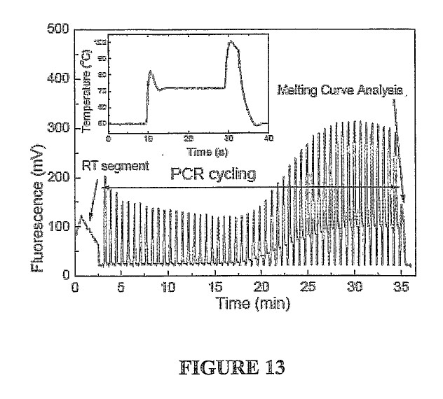

As shown in FIG. 13 (inset), the transition time from one temperature to another was only a few seconds, and the thermal profile is nearly rectangular. The fastest SYBR Green I-based PCR was successfully run at 14.5 s per cycle, while an intrinsically faster approach using FAM as the probe, which is based on thermocycling between two temperatures only, can be run at 8 s per cycle.

FIG. 13: Fluorescence signal of the RT-PCR, followed by a melting curve analysis. The temperature profile of a single PCR cycle is shown as inset, demonstrating fast heating and cooling rates.

The raw fluorescent signal from the optical unit was recorded simultaneously with the temperature and is shown in FIG. 13. The total time for the RT-PCR procedure was around 35 min.

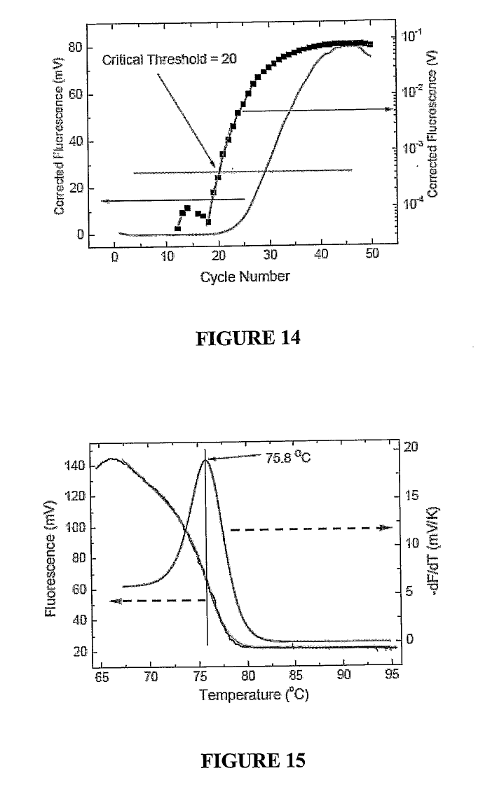

During the PCR cycling, the average amplitude of the fluorescent signal of the last 5 s of the extension segment at 72° C. segment was recorded as a function of the cycle number, normalized against the background, and plotted into a graph, as shown in FIG. 14. A critical threshold C_{T} value was extracted from a graph of the differential fluorescence in logarithmic scale.

FIG. 14: Corrected average fluorescence extracted in last 5 s during the 72° C. segment (red) and differential fluorescence (blue). The total copy number in 20 μL of the solution was 2×10⁶, corresponding to 10⁵ RNA copies in the volume of 1 μL. The extracted value of the critical threshold was 20 cycles, which is in good agreement with the value obtained using a commercial thermocycler (Roche LIGHTCYCLER™ 1.5).

Once the PCR cycling was finished, the melting curve analysis was conducted with a heating rate of 1° C. s⁻¹. The fluorescent signal as a function of temperature was recorded (see FIG. 15). Measured data points were fitted using the following sigmoidal function,

$$y = \frac{( {A}_{0} - x ) ( {A}_{1} - {A}_{2} )}{1 + exp ( \frac{x - {x}_{0}}{k} )} + {A}_{2} + {A}_{3} x ,$$

where A₁, A₂, A₃ are normalization constants, the parameter x₀ represents the location of the inflexion point and k determines the maximum slope at that point. As for the melting curve, the parameter x₀ represents the melting temperature. Typically, the negative value of the first derivative of the melting curve is plotted as a function of temperature to determine the melting temperature.

FIG. 15: Melting curve (see FIG. 13) using a heating rate of 1° C. s⁻¹. The melting temperature, defined as the temperature at which half of the dsDNA is molten/denaturated, was measured to be 75.8° C. This correlates well with the expected value of 76° C., measured by commercial real-time PCR (Roche LIGHTCYCLER™ 1.5) with a ramping rate of 1° C. s⁻¹.

As can be understood by one skilled in the art, many modifications to the exemplary embodiments described herein are possible. The invention, rather, is intended to encompass all such modification within its scope, as defined by the claims.

All documents referred to herein are fully incorporated by reference.

1. A detection system for detecting a fluorescent signal, comprising:
a light source that generates excitation light having a wavelength sufficient to excite a fluorophore in a sample;
an excitation filter positioned along a first line along a path of said excitation light, said excitation filter transmitting the excitation light from the light source;
a beam splitter positioned along said first line, said beam splitter reflecting said excitation light transmitted by said excitation filter along a second line toward a mirror positioned on one side of said beam splitter, and passing emitted light reflected along said second line;
said mirror, positioned to reflect said excitation light from said beam splitter to said fluorophore in said sample along a third line, normal to both said first and second lines, wherein said mirror further reflects emitted light emitted along said third line, along said second line toward said beam splitter;
an emission filter positioned along said second line, on a second side of said beam splitter; and
a detector that detects said emitted light transmitted by said emission filter.

2. The detection system of claim 1, wherein said beam splitter is a dichroic mirror.

3. The detection system of claim 1, wherein said first line is substantially perpendicular to said second line.

4. A detection system for detecting a fluorescent signal, comprising:
a light source that generates excitation light having a wavelength sufficient to excite a fluorophore in a sample;
an excitation filter positioned along a first line along a path of said excitation light, said excitation filter transmitting the excitation light from the light source toward a mirror;
an emission filter positioned along a second line;
said mirror, positioned to reflect said excitation light to said fluorophore in said sample along a third line, normal to both said first and said second lines, wherein said mirror further reflects emitted light emitted along said third line, along said second line toward said emission filter; and
a detector that detects said emitted light transmitted by said emission filter.

5. The detection system of claim 1, further comprising a first lens positioned between said light source and said excitation filter for collimating the excitation light.

6. The detection system of claim 1, further comprising a second lens positioned to focus said excitation light reflect by said mirror toward a said sample.

7. The detection system of claim 1, further comprising an amplifier connected to said detector, for amplifying a signal from said detector.

8. The detection system of claim 1, wherein said light source is an LED.

9. The detection system of claim 8, wherein said LED is a single LED, and said excitation filter and said emission filter are each a single band pass filter.

10. The detection system of claim 8, wherein said LED is a multiple LED, and said excitation filter and said emission filter are each a multiple band pass filter.

11. The detection system of claim 8, further comprising one or more additional single LEDs, wherein said excitation filter and said emission filter are each a multiple band pass filter, and wherein excitation light from each single LED is individually modulated and demodulated.

12. The detection system of claim 1, wherein said detector is a photodiode.

13. A thermocycler device comprising:
a detection system as defined in claim 1;
a sample port for receiving a sample containing a fluorophore, said sample port positioned to place said sample in line with an excitation light reflected from said detection system;
a heater positioned adjacent to said sample receiving port for heating said sample;
a temperature sensor connected to said heater for detecting said temperature of said heater;
a fluorescent signal processor connected to said detection system for processing a fluorescent signal detected by said detection system;
a user interface module for input and output of data; and
a power source for powering said device.

14. The thermocycler device of claim 13, further comprising a controller in communication with said heater, said temperature sensor, said fluorescent signal processor and said user interface module.

15. The thermocycler device of claim 13, wherein said user interface module comprises a touch screen.

16. The thermocycler device of claim 13, wherein said thermocycler device is a hand-held device.

17. The thermocycler device of claim 13, wherein said power source is a battery.

18. The detection system of claim 4, further comprising a first lens positioned between said light source and said excitation filter for collimating the excitation light.

19. The detection system of claim 4, further comprising a second lens positioned to focus said excitation light reflect by said mirror toward a said sample.

20. The detection system of claim 4, further comprising an amplifier connected to said detector, for amplifying a signal from said detector.

21. The detection system of claim 4, wherein said light source is an LED.

22. The detection system of claim 21, wherein said LED is a single LED, and said excitation filter and said emission filter are each a single band pass filter.

23. The detection system of claim 21, wherein said LED is a multiple LED, and said excitation filter and said emission filter are each a multiple band pass filter.

24. The detection system of claim 21, further comprising one or more additional single LEDs, wherein said excitation filter and said emission filter are each a multiple band pass filter, and wherein excitation light from each single LED is individually modulated and demodulated.

25. The detection system of claim 4 wherein said detector is a photodiode.

26. A thermocycler device comprising:
a detection system as defined in claim 4;
a sample port for receiving a sample containing a fluorophore, said sample port positioned to place said sample in line with an excitation light reflected from said detection system;
a heater positioned adjacent to said sample receiving port for heating said sample;
a temperature sensor connected to said heater for detecting said temperature of said heater;
a fluorescent signal processor connected to said detection system for processing a fluorescent signal detected by said detection system;
a user interface module for input and output of data; and
a power source for powering said device.

27. The thermocycler device of claim 26, further comprising a controller in communication with said heater, said temperature sensor, said fluorescent signal processor and said user interface module.

28. The thermocycler device of claim 26, wherein said user interface module comprises a touch screen.

29. The thermocycler device of claim 26, wherein said thermocycler device is a hand-held device.

30. The thermocycler device of claim 26, wherein said power source is a battery.
